(19) United States
(12) Patent Application Publication
(10) Pub. No.: US 2010/0191171 A1
(43) Pub. Date: Jul. 29, 2010

(54) Iontophoresis device

(75) Inventor: Hyeon-Woo Park, Daejeon-City (KR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: LG Household & Health Care Ltd., Seoul (KR)

(21) Appl. No.: 12/310,584

(22) PCT Filed: Aug. 29, 2007
(86) PCT No.: PCT/KR2007/004158

§ 371 Date: Feb. 27, 2009

(30) Foreign Application Priority Data
Aug. 30, 2006 (KR) 10-2006-0083057
Nov. 29, 2006 (KR) 10-2006-0118999
Jul. 6, 2007 (KR) 10-2007-0068180
Aug. 24, 2007 (KR) 10-2007-0085302

(51) Int. Cl. A61N 1/30

(52) U.S. Cl. 604/20

(57) ABSTRACT

The iontophoresis device according to the exemplary embodiment of the present invention may concentrate care skin to where liver spots and freckles exist, and may be compact, portable, and convenient. An iontophoresis device according to an exemplary embodiment of the present invention may include: a housing providing an inner space; a battery installed in the housing for supplying power; a control circuit for controlling a current output from the battery; a first contact portion electrically connected with the battery and contacting the skin; a second contact portion electrically connected with the battery and disposed to the housing; and an operation portion for turning the power on and off.

### CROSS-REFERENCE TO RELATED APPLICATION

This application claims priority to and the benefit of Korean Patent Application No. 10-2006-0083057 filed in the Korean Intellectual Property Office on Aug. 30, 2006, Korean Patent Application No. 10-2006-0118999 filed in the Korean Intellectual Property Office on Nov. 29, 2006, Korean Patent Application No. 10-2007-0068180 filed in the Korean Intellectual Property Office on Jul. 6, 2007, and Korean Patent Application No. 10-2007-0085302 filed in the Korean Intellectual Property Office on Aug. 24, 2007, the entire contents of which are incorporated herein by reference.

### BACKGROUND OF THE INVENTION

(a) Field of the Invention

The present invention relates to an iontophoresis device. More particularly, the present invention relates to the iontophoresis device which may easily carry an active material and uses the iontophoresis theory with a small current.

(b) Description of the Related Art

In order for an active material or cosmetics that include a moisturizer or other materials for skin care, particularly for facial skin care, to be deeply absorbed into skin, a method is used in which a mask pack including a non-woven fabric with an active material or cosmetics spread thereon is adhered to the skin for some time.

However, the conventional method that uses a mask pack cannot provide sufficient results or skin absorption effect.

For improving this, a method in which a small current flows to the skin so that a charged material or other effective elements can penetrate the skin is used with the mask pack. However, the mask pack has problems in that the adhering characteristic is not good because of irregularities of skin, and it is painful when the mask pack is separated from the skin.

Meanwhile, a skin care apparatus in which the iontophoresis theory is applied has been used, but it is heavy and non-portable. In addition, the skin care apparatus is used for an active material to penetrate large portions of the skin so it is difficult to apply for spot care.

A conventional skin care apparatus is used after manually coating skin with active materials, so it is uncomfortable.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide an iontophoresis device having advantages of compactness, portability, and the potential to protect against skin trouble such as liver spots, freckles, and wrinkles.

The iontophoresis device is connected to a first contacting portion and improves skin problems such as liver spots, freckles, or wrinkles. The iontophoresis device may reduce inconvenience of skin care after coating the skin with active materials manually or with other instruments.

An iontophoresis device according to an exemplary embodiment of the present invention may include: a housing providing an inner space; a battery installed in the housing for supplying power; a control circuit for controlling a current output from the battery; a first contact portion electrically connected with the battery and contacting skin; a second contact portion electrically connected with the battery and disposed to the housing; and an operation portion for turning the power on and off.

The iontophoresis device may include a storage portion installed to an end of the housing for filling with an active material. The first contact portion may be configured to the storage portion, and the iontophoresis device may include a discharging means for discharging the active material filled in the storage portion to the outside thereof.

The storage portion may include an outer case, and a storage tank that is made of a transparent material and that is configured inside of the outer case.

The storage portion may include an electrically conductive layer that is electrically connected to the first contact portion between the outer case and the storage tank.

The storage tank may be separated from the outer case and the electrically conductive layer.

The storage tank may be made of a glass material.

The outer case may include a display unit, which displays the left amount of the active material in the storage tank, and the display unit may be formed by eliminating a part of the outer case in a slot shape.

The outer case may be made of an insulating material.

The discharging means may be a wick that penetrates the first contact portion, absorbs the active material filled in the storage tank, and releases the active material to the outside thereof.

The discharging means may be an airless pump configured to an opening of the storage tank.

A cap that is assembled or disassembled with the storage portion for protecting the first contact portion may be formed.

The iontophoresis device may further include a ball that functions with the discharging means and the first contact portion.

The battery may be a coin-type battery, a seat-type battery, or a cylindrical battery.

An opening/closing portion may be formed in the housing for replacing the battery.

The current control circuit may have a function of controlling a level of the current flowing from the battery to the skin of a user through the first contact portion.

The iontophoresis device may further include an indicating portion that indicates an on/off state of the power and the level of the current.

The current control circuit may have a function through which a pulse-type current can flow to the skin of a user, and a frequency of the pulse-type current is 1000 to 50,000 Hz.

The first contact portion may be made of stainless steel, and a coating layer made of titanium nitride (TiN) may be formed on a surface of the first contact portion.

The material, which may have a skin lightening effect, may be at least one of arbutin, vitamin C (ascorbic acid), MAP (magnesium ascorbyl phosphate), and AA-2G (ascorbyl acid-2 glucoside).

The material, which may have a wrinkle care effect, may be at least one of vitamin C (ascorbic acid), MAP (magnesium ascorbyl phosphate), AA-2G (ascorbyl acid-2 glucoside), paeoniflorin, kinetin, an auxin, a peptide, and an alpha-hydroxy acid.

An iontophoresis device according to an exemplary embodiment of the present invention may include: a housing providing an inner space; a battery installed in the housing for supplying power; a control circuit for controlling a current output from the battery; a first contact portion electrically connected with one electrode of two electrodes of the battery and contacting the skin; a second contact portion electrically connected with the other electrode of the battery and disposed to the housing; and an operation portion for turning the power on and off, wherein the first contact portion, which is formed to be assembled and disassembled, may be mounted to an end of the housing.

The housing may have a bar shape, and the housing may form a protrusion portion at the end where the first contact portion connects thereto.

The protrusion portion may be cylindrical in shape, and a male thread may be formed on a circumferential surface of the protrusion portion.

A female thread corresponding to the male thread of the protrusion portion may be formed on an inner surface of the first contact portion, and the first contact portion may be connected with the protrusion portion by screw coupling. The first contact portion may be provided with an opening at an end of the first contact portion, and the first contact portion may include a ball that is configured in the opening and spins.

An electrically conductive layer may be formed on an inner surface of the first contact portion, and the first contact portion may be made of a conductor.

The first contact portion may include a main body portion on which a female thread corresponding to the male thread of the protrusion portion is formed on an inner surface thereof and that is connected with the protrusion portion by screw coupling, and a head portion that is connected to the main body portion and massages the skin.

An electrically conductive layer may be formed on an inner surface of the main body portion, and the main body portion may be made of a conductor. The main body portion and the head portion may be integrally formed.

The first contact portion may include a covering portion identically formed with the male thread of the protrusion portion, and a body portion of which an upper part connecting with the covering portion is opened, that has an inner space in which a female thread corresponding to the male thread of the covering portion is formed on an inner surface thereof, and that is connected with the covering portion by screw coupling.

The protrusion part may be connected to a lower surface of the body portion.

The protrusion part may be cylindrical in shape with an opening at an end thereof, and the first contact portion may include a ball that is configured to spin at the opening. A male thread may be formed on an external circumferential surface of the protrusion part. A female thread corresponding to the male thread of the protrusion part may be formed, and the iontophoresis device may further include a protecting cap that is connected with the protrusion part by screw coupling.

The body portion may be made of one among glass, acrylonitrile-butadiene-styrene (ABS), polyethylene (PE), polyethylene terephthalate (PET), and a conductive plastic. The body portion may be made of glass, and an inner side of the body portion may be made of a conductive plastic.

The iontophoresis device may further include a head portion that is connected to the protecting cap and massages the skin. An electrically conductive layer may be formed on an inner surface of the protecting cap. The protecting cap and the head portion may be integrally formed.

An iontophoresis device according to an exemplary embodiment of the present invention may further include a storage portion installed in the housing for filling the active material therein, and the first contact portion may include a ball contact portion that is connected with an end of the storage portion and forms a path where the active material filled in the storage portion is released to the outside thereof, and a cap contact portion that is shaped to cover the ball contact portion, is connected with the storage portion, and massages the skin with a surface thereof.

The ball contact portion may include a connecting portion with an end thereof connected with the storage portion, a ball that is configured to an opening to spin where the opening is formed at the other end of the connecting portion, and an elastic portion that is disposed between an end of the storage portion and the ball and that supports the ball in the connecting portion.

The storage portion may include a protrusion portion that is formed in a cylindrical shape by protruding a part connected to the connecting portion, and the protrusion portion may have an opening to form a path to flow the active material to the outside thereof.

The iontophoresis device may further include a guide portion that is safely disposed on the opening of the protrusion portion and supports the elastic portion. The guide portion may include a bottom portion having a groove where the elastic portion is safely disposed, and the bottom portion may have a hole for the active material to be released. A blocker may be formed on a circumference of the guide portion, and a diameter of the blocker may be larger than the internal diameter of the opening of the protrusion portion.

A cap contact portion may be made of a conductor and may have a function of iontophoresis when massaging skin.

### BRIEF DESCRIPTION OF THE DRAWINGS

FIG. 1 is a schematic cross-sectional view of an iontophoresis device according to the first exemplary embodiment of the present invention.

FIG. 2 is a schematic cross-sectional view of an iontophoresis device according to the second exemplary embodiment of the present invention

FIG. 3 is a schematic cross-sectional view of an iontophoresis device according to the third exemplary embodiment of the present invention.

FIG. 4 is a schematic cross-sectional view of an iontophoresis device according to the fourth exemplary embodiment of the present invention.

FIG. 5 is a schematic cross-sectional view of an iontophoresis device according to the fifth exemplary embodiment of the present invention.

FIG. 6 is a schematic cross-sectional view of an iontophoresis device according to the sixth exemplary embodiment of the present invention.

FIG. 7 is a view showing the connecting relationship of the first contact portion of FIG. 6.

FIG. 8 is a view showing the connecting relationship of the first contact portion according to the seventh exemplary embodiment of the present invention.

FIG. 9 is a view showing the connecting relationship of the first contact portion according to the eighth exemplary embodiment of the present invention.

FIG. 10 is a cross-sectional view along a line X-X of FIG. 9.

FIG. 11 is a view showing the connecting relationship of the first contact portion according to the ninth exemplary embodiment of the present invention.

FIG. 12 is a cross-sectional view along a line X II-X II of FIG. 11.

FIG. 13 is a view showing the connecting relationship of the first contact portion according to the tenth exemplary embodiment of the present invention.

FIG. 14 is a view showing a separated state of the cap contact portion of FIG. 13.

FIG. 15 is a view showing a connected state of the cap contact portion of FIG. 13.

FIG. 16 is a view showing the ball contact portion of FIG. 13.

### DETAILED DESCRIPTION OF THE EMBODIMENTS

The present invention will be described more fully hereinafter with reference to the accompanying drawings, in which exemplary embodiments of the invention are shown. While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims. In the specification and drawings, the same reference numbers will indicate the same elements.

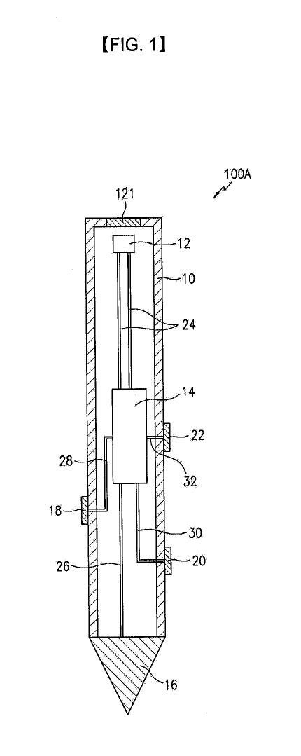

Referring to FIG. 1, of an iontophoresis device 100A according to the first exemplary embodiment of the present invention includes a housing 10, a battery 12, a current control circuit 14, a first contact portion 16, a second contact portion 18, an adjusting portion 20, and an indicating portion 22.

The housing 10 is a main body of the iontophoresis device 100A and is formed in a bar shape for convenience of gripping and portability, and is provided with a space for the battery 12 and the current control circuit 14. An opening/closing portion 121 is formed on an end of the housing 10 for replacing the battery 12.

The housing 10 may be made of an insulating material for insulating the first contact portion 16 and the second contact portion 18 from each other, and it may has a shape of a circle, a polygon, or so on in cross section.

The battery 12 may be an arbitrary battery that outputs electrical energy by using chemical energy. The battery 12 may be a small and light battery, for example a coin-type battery, a seat-type battery, or a cylindrical battery. The iontophoresis device may be light and small as a whole because a coin-type battery, a seat-type battery, or a cylindrical battery is used.

The current control circuit 14 is electrically connected to the battery 12 through a first connecting line 24, and controls a current outputted from the battery 12. The current control circuit 14 may have a function that can control a level of the current flowing from the battery 12 to the skin of a user through the first contact portion.

The current control circuit 14 may have a function such that a pulse-type current can flow to the skin of a user. Because the pulse-type current flows, polarization of the skin may reduced so that erythema or burns may be minimized. For example, a frequency of the pulse-type current may be 1000 to 50,000 Hz. If a high frequency current flows, impedance in the skin is lowered so that resistance is reduced, but the depth of drug transmission is lowered. On the contrary, if a low frequency current flows, the depth of drug transmission is increased, but resistance of the skin is increased such that the amount of drug transmission is reduced. Accordingly, the optimum frequency range is 1000 to 50,000 Hz.

The current control circuit 14 may have a current supplying interval and a current interruption interval. The sum of the current supplying interval and the current interruption interval with respect to the current supplying interval as a ratio is a duty cycle, in which an idle period when current is not transmitted to the skin occurs so that polarization can be minimized. The duty cycle may be adjusted according to the level of current or the kind of drug.

The current control circuit 14 is obvious to the person skilled in the art, so a detailed description thereof will be omitted.

The first contact portion 16 is electrically connected to one of the two electrodes of the battery 12 through the first connecting line 24 and a second connecting line 26, and contacts the skin directly. The first contact portion 16 contacts the skin where a user desires care, and may be mounted to an end of the housing 10. The first contact portion 16 may be formed to contact a part of the skin by spot contact or line contact.

The first contact portion 16 may be made of high quality stainless steel for electrical conductivity and skin safety, and a coating layer made of titanium nitride (TiN) may be formed on a surface of the first contact portion for preventing skin allergy.

The second contact portion 18 is electrically connected to the other of the two electrodes of the battery 12 through the first connecting line 24 and a third connecting line 28. The second contact portion 18 directly contacts the hand of the user when the user applies the iontophoresis device 100A by hand.

The adjusting portion 20 may be a button configured to switch ON/OFF and to control the current control circuit 14, and may be connected to the current control circuit 14 through a fourth connecting line 30. As an example, if the adjusting portion 20 is pressed once, the power turns on and a low current flows, if the adjusting portion 20 is pressed twice, then a mid-level current flows, if the adjusting portion 20 is pressed three times, then a high current flows, and if the adjusting portion 20 is pressed four times, the power turns off.

The indicating portion 22 indicates the state of the iontophoresis device 100A according to the signal of the adjusting portion 20, it may be a lamp such as a light emitting diode (LED), and the indicating portion 22 is connected to the current control circuit 14 through a fifth connecting line 32. As an example, if the adjusting portion 20 is pressed once, the lamp exhibits a green color, if the adjusting portion 20 is pressed twice, the lamp exhibits an orange color, if the adjusting portion 20 is pressed three times, the lamp exhibits a red color, and if the adjusting portion 20 is pressed four times, the lamp turns off.

The iontophoresis device 100A according to the exemplary embodiment of the present invention forms a closed electrical circuit connecting the battery 12, the current control circuit 14, the first contact portion 16, a body of the user, the second contact portion 18, and the battery 12.

Accordingly, the current flows from the battery 12 to the body of the user, so that absorption of the active material by the skin may be promoted by the iontophoresis effect.

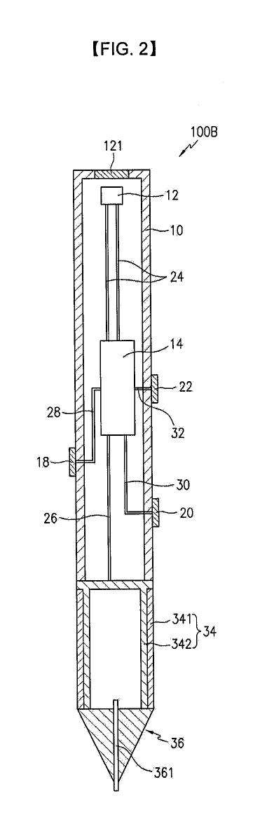

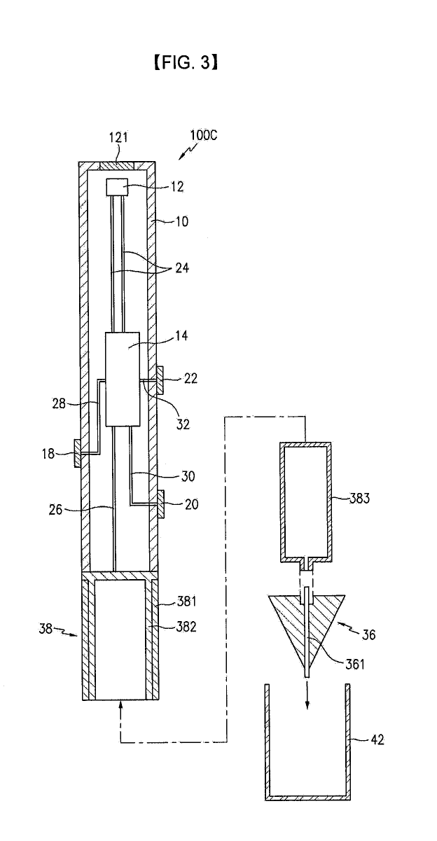

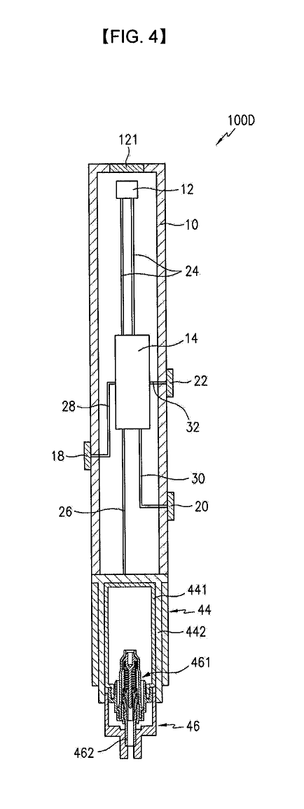

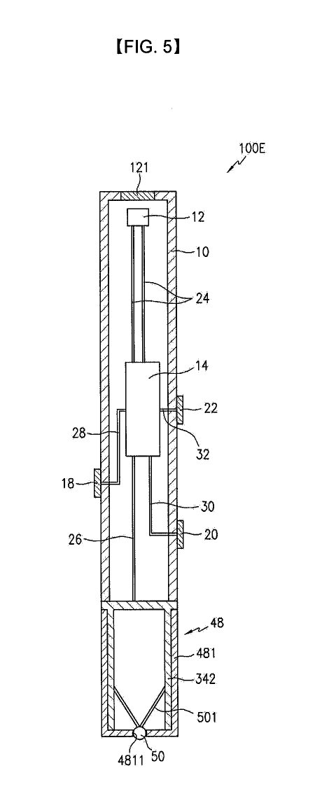

FIG. 2 is a schematic cross-sectional view of an iontophoresis device according to the second exemplary embodiment of the present invention; FIG. 3 is a schematic cross-sectional view of an iontophoresis device according to the third exemplary embodiment of the present invention; FIG. 4 is a schematic cross-sectional view of an iontophoresis device according to the fourth exemplary embodiment of the present invention; and FIG. 5 is a schematic cross-sectional view of an iontophoresis device according to the fifth exemplary embodiment of the present invention, and all show the iontophoresis device including a storage portion for filling with an active material.

The same reference numbers as used in the first exemplary embodiment will indicate the same elements, and detailed descriptions thereof will be omitted.

Referring to FIG. 2, an iontophoresis device 100B according to an exemplary embodiment of the present invention includes a storage portion 34 installed to an end of the housing 10 for filling with the active material. The storage portion 34 is electrically connected to an outer case 341 made of an insulating material and a second connecting line 26, and the storage portion 34 includes an electrically conductive layer 342 closely formed to the outer case 341.

A first contact portion 36 is electrically connected to the electrically conductive layer 342 and mounted to an end of the storage portion 34. The first contact portion 36 is provided with a discharging means for discharging the active material filled in the storage portion 34 to the outside thereof. The discharging means may be a wick 361. The wick 361 penetrates the first contact portion 36 and protrudes to the outside thereof such that one end of the wick 361 contacts the active material filled in the storage portion 34 and the other end of the wick 361 contacts the skin.

The wick 361 may be made of a non-woven fabric so that the wick 361 may exude the active material when it is pressed after absorbing the active material.

The active material may include the material that has a lightening effect. For example, the material that has the skin lightening effect may be at least one of arbutin, vitamin C (ascorbic acid), MAP (magnesium ascorbyl phosphate), and AA-2G (ascorbyl acid-2 glucoside). The MAP and the AA-2G are vitamin C derivatives.

The active material may include a material that has a wrinkle care effect. For example, the material that has a wrinkle care effect is at least one of vitamin C (ascorbic acid), MAP (magnesium ascorbyl phosphate), AA-2G (ascorbyl acid-2 glucoside), paeoniflorin, kinetin, an auxin, a peptide, and an alpha-hydroxy acid.

It is preferable that the peptide includes 10 or fewer amino acids. Absorption is deteriorated in spite of the iontophoresis effect if the peptide includes many amino acids. Particularly, it is preferable that the peptide include 2 through 6 amino acids, because that number of amino acids makes absorption sufficient.

The iontophoresis device 100B according to an exemplary embodiment of the present invention includes the storage portion 34 and releases the active material to the skin through the discharging means so that it is convenient to use.

Referring to FIG. 3, an iontophoresis device 100C according to an exemplary embodiment of the present invention includes a storage portion 38 that can be separated from the housing 10. The storage portion 38 includes an outer case 381 made of an insulating material, an electrically conductive layer 382 that is electrically connected to a second connecting line 26 and closely formed inside of the outer case 381, and a storage tank 383 that is disposed in the outer case 381 and the electrically conductive layer 382.

The storage tank 383 can be separated from the outer case 381 and the electrically conductive layer 382 and replaced when the active material in the storage tank 383 is exhausted.

The storage tank 383 may be provided with an opening in order for a wick 361 of the first contact portion 36 to be inserted therein, and may be made of a transparent material such as glass.

The outer case 381 and the electrically conductive layer 382 may be provided with a display unit, which displays the left amount of the active material filled in the storage tank 383. For example, the display unit can be formed on the outer case 381 and the electrically conductive layer 382 by making groove shaped as a slot along the vertical direction.

The iontophoresis device 100C according to an exemplary embodiment of the present invention may further include a cap 42 that is assembled or disassembled with the storage portion 38 or the housing 10 for protecting the first contact portion 36.

Referring to FIG. 4, an iontophoresis device 100D according to an exemplary embodiment of the present invention includes a first contact portion 46 that is provided with an airless pump 461 as a discharging means for discharging the active material from a storage portion 44.

The airless pump 461 is configured to an opening of the storage tank 441 and releases the active material in the storage tank 441 by pressure of a shaft 462. The shaft 462 reciprocally moves by pressure of the storage tank 441 that is electrically connected to the electrically conductive layer 442 of the storage portion 44.

The airless pump is a device for discharging a fluid according to a change of pressure by reciprocal motion. A conventional airless pump can be applied.

FIG. 5 is a schematic cross-sectional view of an iontophoresis device according to the fifth exemplary embodiment of the present invention. An iontophoresis device 100E according to the fifth exemplary embodiment of the present invention is similar to the iontophoresis device 100B of FIG. 2, and the same reference numbers of the exemplary embodiment will indicate the same elements and a detailed description thereof will be omitted.

Referring to FIG. 5, the iontophoresis device 100E according to an exemplary embodiment of the present invention includes a ball 50 as a discharging means. The storage portion 48 includes an outer case 481 and an electrically conductive layer 342, and the active material is filled in a storage portion 48. The outer case 481 is made of an insulating material. The electrically conductive layer 342 is electrically connected to the second connecting line 26.

An opening 4811 is formed at one end of the storage portion 48, and the ball is configured to the opening 4811. The active material in the storage portion 48 is released through the ball 50. If the ball 50 is rubbed on the skin, friction is generated between the ball 50 and the skin. The friction makes the ball 50 spin, and the spinning ball 50 causes the active material in the storage portion 48 to be spread over the skin. The ball 50 is electrically connected to the electrically conductive layer 342 of the storage portion 48. For the connection, a conductive connecting portion 501 is disposed. One end of the connecting portion 501 is connected to the electrically conductive layer 342, and the other end thereof contacts the ball 50.

The iontophoresis device 100E configures the ball 50 to one end of the storage portion 48 so that no contacting means are needed. Accordingly, the structure of the apparatus is simplified. The active material can be released to the skin through the spherical surface of the ball 50, so that skin trouble may be minimized.

The iontophoresis device 100E according to an exemplary embodiment of the present invention, as described in FIG. 3, may further include a cap 42 that is assembled or disassembled with the storage portion 48 or the housing 10 for protecting the opening 4811 and the ball 50.

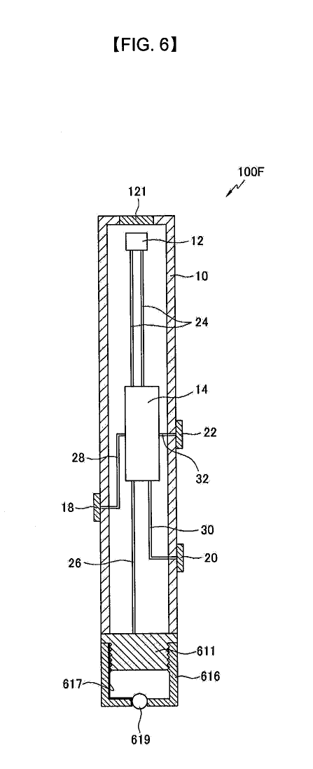

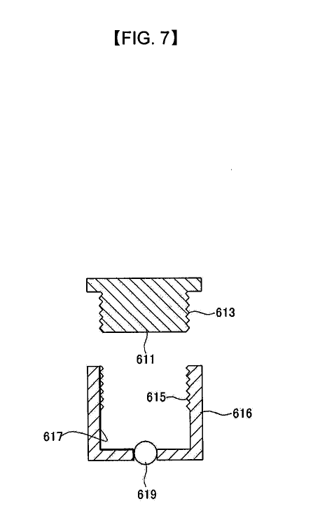

Referring to FIG. 6 and FIG. 7, according to a sixth exemplary embodiment of the present invention of an iontophoresis device 100F, a first contact portion 616 is assembled or disassembled with the housing 10, and the iontophoresis device 100F has a space for filling with the active material.

The housing 10 has a bar shape, and a protrusion portion 611 is formed on one end that is connected to the first contact portion 616. The protrusion portion 611 has a cylindrical shape, and a male thread 13 is formed on a circumferential surface of the protrusion portion 611.

A female thread 15 corresponding to the male thread 13 of the protrusion portion 611 is formed on an inner surface of the first contact portion 616, and the first contact portion 616 is connected with the protrusion portion 611 by screw coupling. Accordingly, the first contact portion 616 is assembled or disassembled with the housing 10.

The first contact portion 616 is provided with an opening at an end thereof, and includes a ball 619 that is configured in the opening and spins. The ball 619 is a discharging means for discharging the active material from the first contact portion 616. The active material in the first contact portion 616 is released by means of the ball 619. If the ball 619 is rubbed on the skin, friction is generated between the ball 619 and the skin. The friction makes the ball 619 spin, and the spinning ball 619 causes the active material in the first contact portion 616 to be spread over the skin. The ball 619 is electrically connected to an electrically conductive layer 617 of a first contact portion 616.

The iontophoresis device 100F according to the sixth exemplary embodiment of the present invention configures the ball 619 to one end of the first contact portion 616 so that no contacting means are needed. Accordingly, the structure of the apparatus is simplified. The active material can be released to the skin through the spherical surface of the ball 619, so that skin trouble may be minimized.

The electrically conductive layer 617 is formed on an inner surface of the first contact portion 616 and the first contact portion 616 is made of a conductor as a whole. When the electrically conductive layer 617 is formed on an inner surface of the first contact portion 616, an outer surface of the first contact portion 616 may be made of an insulating material. The electrically conductive layer 617 is electrically connected to the second connecting line 26.

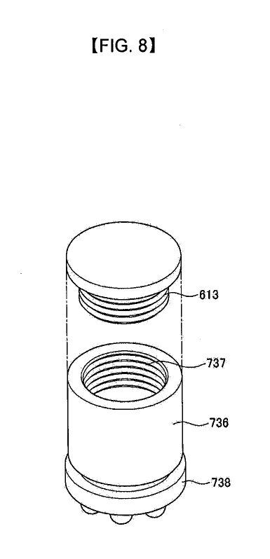

Referring to FIG. 8, a first contact portion according to a seventh exemplary embodiment of the present invention includes a main body portion 736 and a head portion 738.

A female thread 737, corresponding to the male thread 13 of the protrusion portion 611, is formed on an inner surface of the main body portion 736 and is connected with the protrusion portion 611 by screw coupling. An electrically conductive layer is formed on an inner surface of the main body portion 736, and the main body portion 736 may be made of a conductor as a whole.

The head portion 738 is connected to the main body portion 736 and massages the skin. The head portion 738 may be formed in a semi-spherical shape by one or a plurality of balls, and the shape can be changed according to need.

The main body portion 736 and the head portion 738 may be integrally formed.

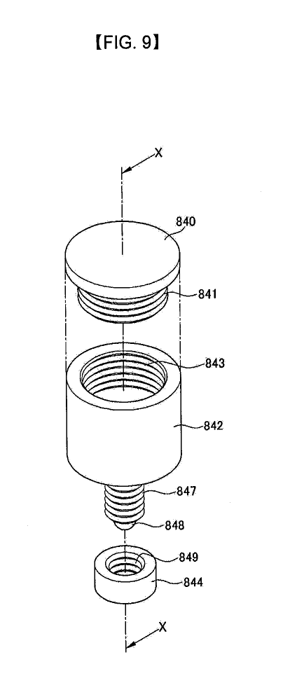

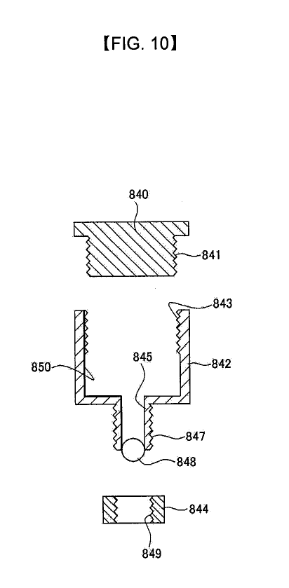

Referring to FIG. 9, the first contact portion according to the eighth exemplary embodiment of the present invention may include a covering portion 840, a body portion 842, and a protecting cap 844, and may be made as an ampule. FIG. 10 is a cross-sectional view along a line X-X of FIG. 9, and shows a screw connecting relationship between the covering portion 840 and the body portion 842, and between a protrusion part 845 and the protecting cap 844.

The covering portion 840 has a male thread 841 identical to the male thread 13 of the protrusion portion 611. The covering portion 840 is connected to the body portion 842, and is separated from the body portion 842 when the body portion 842 is connected to the protrusion portion 611 of the housing 10.

The body portion 842, of which an upper part is opened when the covering portion 840 is disconnected therefrom, has an inner space and a female thread 843 corresponding to the male thread 841 of the covering portion 840 formed on an inner surface thereof, and is connected with the covering portion 840 by screw coupling. The body portion 842 may be made of one of glass, acrylonitrile-butadiene-styrene (ABS), polyethylene (PE), polyethylene terephthalate (PET), and a conductive plastic.

The protrusion part 845 is connected to a lower surface of the body portion 842.

The protrusion part 845 is cylindrical in shape and has an opening at an end thereof, and the first contact portion includes a ball 848 that is configured to spin at the opening. A male thread 847 is formed on an external circumferential surface of the protrusion part 845.

The body portion 842 is made of glass, and an inner side of the body portion 842 is made of a conductive plastic. The conductive plastic may be formed from the female thread 843 formed in the body portion 842 to the opening contacting the ball 848.

An electrically conductive layer 850 may be formed on an inner surface of the body portion 842. When the electrically conductive layer 850 is formed on an inner surface of the body portion 842, an outer surface of the body portion 842 may be made of an insulating material. The electrically conductive layer 850 is electrically connected to the second connecting line 26.

A female thread 849 corresponding to the male thread 847 of the protrusion part 845 is formed in the protecting cap 844, which is connected with the protrusion part 845 by screw coupling. The protecting cap 844 may be assembled or disassembled with the first contact portion, and serves to protect the opening of the first contact portion and the ball 848.

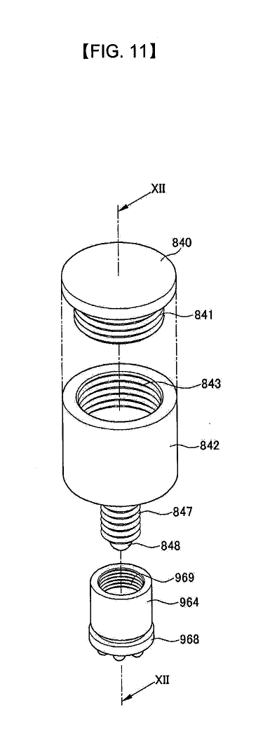

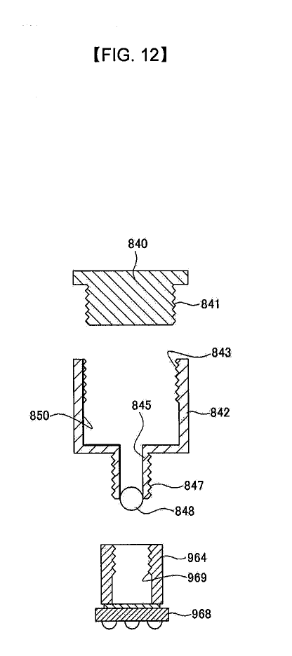

Referring to FIG. 11, the first contact portion according to the ninth exemplary embodiment of the present invention may include a covering portion 840, a body portion 842, and a protecting cap 964, and may be made as an ampule. FIG. 12 is a cross-sectional view along a line X II-X II of FIG. 11, and shows a screw connecting relationship between the covering portion 840 and the body portion 842, and between a protrusion part 845 and the protecting cap 964.

The configuration and connecting relationship of the covering portion 840 and the body portion 842 may be identical to the eighth exemplary embodiment of the present invention.

A female thread 969 corresponding to the male thread 847 of the protrusion part 845 is formed on an upper part of the inner surface of the protecting cap 964, which is connected with the protrusion part 845 by screw coupling. The protecting cap 964 may be assembled or disassembled with the first contact portion for protecting the opening of the first contact portion and the ball 848.

A head portion 968 is formed on the lower part of the protecting cap 964. The head portion 968 and the protecting cap 964 are integrally formed, and massage the skin. The head portion 968 may be formed in a semi-spherical shape by one or a plurality of balls, and the shape can be changed according to need. An electrically conductive layer is formed on an inner surface of the protecting cap 964, and the protecting cap 964 may be made of a conductor as a whole. The protecting cap 964 and the head portion 968 may be integrally formed.

As described above, the head portion 968 is formed on the protecting cap 964, so that dispensing the active material and massaging can be undertaken separately. For example, the active material can be dispensed to the skin by the ball 848 of the body portion 842. Then, the protecting cap 964 is connected to the protrusion portion 845 of the body portion 842 by screw coupling, and massaging can be performed using the head portion 968.

The protecting cap 964 can be used as the head portion 968. The protecting cap 964 is electrically disconnected to the second contact portion 18, and is electrically connected to the first contact portion 16 or the ball 848.

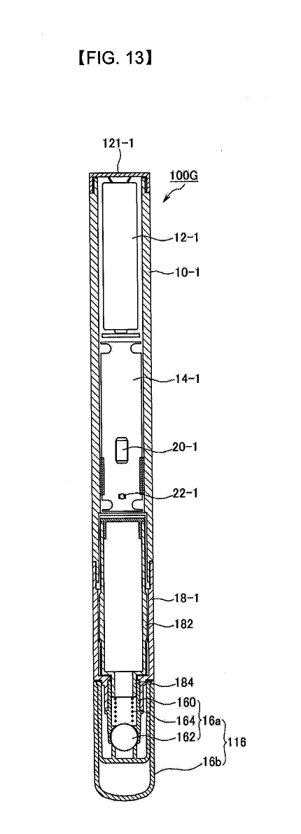

Referring to FIG. 13, the iontophoresis device according to the tenth exemplary embodiment of the present invention includes a housing 10-1, a battery 12-1, a current control circuit 14-1, a first contact portion 116, a storage portion 182, a second contact portion 18-1, an adjusting portion 20-1, and an indicating portion 22-1.

Similar reference numbers to those of the first exemplary embodiment will indicate similar elements, and detailed description thereof will be omitted.

The housing 10-1 is a main body of the iontophoresis device 100G and an opening/closing portion 121-1 for replacing the battery 12-1 is formed on an end thereof.

The first contact portion 116 includes a ball contact portion 16a and a cap contact portion 16b.

The contact portion 16a includes a connecting portion 160, a ball 162, and an elastic portion 164, and the contact portion 16a is connected to an end of the storage portion 182 of the housing 10-1. The ball contact portion 16a forms a path where the active material filled in the storage portion 182 is released to the outside thereof. The ball contact portion 16a is made of a conductor. It is described above that the ball contact portion 16a is connected to the end of the storage portion 182, but the ball contact portion 16a may be mounted or dismounted with the housing 10-1 according to need.

An end of the connecting portion 160 is connected to the storage portion 182.

The ball 162 is configured to an opening to spin where the opening is formed at the other end of the connecting portion 160. The ball 162 is a discharging means for discharging the active material filled in the storage portion 182.

The elastic portion 164 is disposed between an end of the storage portion 182 and the ball 162, and supports the ball 162 in the connecting portion 160. The elastic portion 164 supplies stable pressure to the ball 162 disposed on the opening of the connecting portion 160. For example, if user presses the ball 162 on the skin, the ball 162 presses the elastic portion 164 by the same pressure. The active material is released through a gap between the ball 162 and the connecting portion 160, and is dispensed to the skin.

The first contact portion 116 is electrically connected to the battery 12-1 and contacts the skin directly. The first contact portion 116 contacts the skin directly where the user wants skin care, and is mounted to an end of the housing 10-1. The first contact portion 116 may be formed to contact a part of the skin by spot contact or line contact.

The first contact portion 116 may be made of high quality stainless steel for electrical conductivity and skin safety, and a coating layer made of titanium nitride (TiN) may be formed on a surface of the first contact portion for preventing skin allergy.

The storage portion 182 includes a protrusion portion 184 that is formed in a cylindrical shape by protruding a part connected to the connecting portion 160, and the part is inserted into the connecting portion 160.

The protrusion portion 184 has an opening 185 to form a path to flow the active material to the outside thereof.

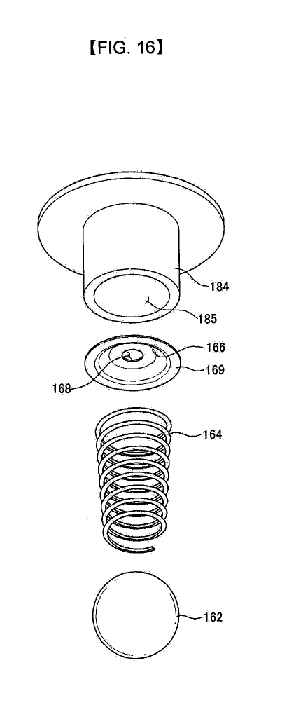

Referring to FIG. 16, the iontophoresis device further includes a guide portion 166 that is safely disposed on the opening 185 of the protrusion portion 184 and supports the elastic portion 164. The guide portion 166 includes a bottom portion having a groove where the elastic portion 164 is safely disposed, and the bottom portion has a hole 168 for the active material to be released. A blocker 169 is formed on a circumference of the guide portion 169. A diameter of the blocker 169 is bigger than an internal diameter of the opening 185 of the protrusion portion 184. Accordingly, the guide portion 166 may not be inserted into the opening 185 of the protrusion portion 184.

As stated above, if the ball 162 is rubbed to the skin, friction is generated between the ball 162 and the skin. The friction makes the ball 162 spin, and the spinning ball 162 causes the active material in the storage portion 182 to be spread over the skin. The ball 162 is electrically connected to the electrically conductive layer of the storage portion 182. For the connection, a conductive connecting line or a conductive connecting portion may be disposed. One end of the connecting portion may be connected to the electrically conductive layer, and the other end thereof may contact the ball 162.

The iontophoresis device 100G according to the tenth exemplary embodiment of the present invention configures the ball contacting portion 16a to one end of the storage portion 182 so that no contacting means are needed. Accordingly, the structure of the apparatus is simplified. The active material can be released to the skin through a spherical surface of the ball 162, so that skin trouble may be minimized.

The iontophoresis device 100G according to the exemplary embodiment of the present invention further includes a cap contact portion 16b that is assembled or disassembled with the storage portion 182 or the housing 10-1 for protecting the first contact portion. The cap contact portion 16b is made of a conductor. Because the cap contact portion 16b is made of a conductor, the iontophoresis effect may be achieved when massaging skin.

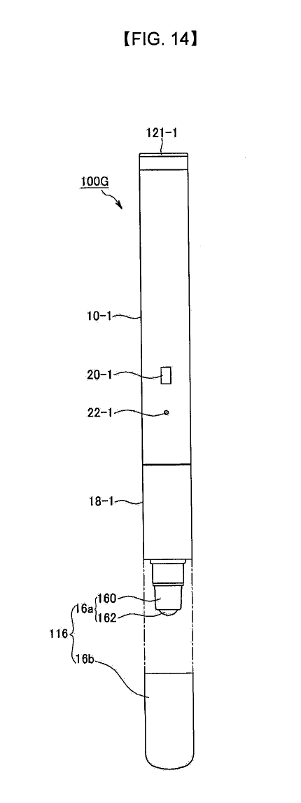

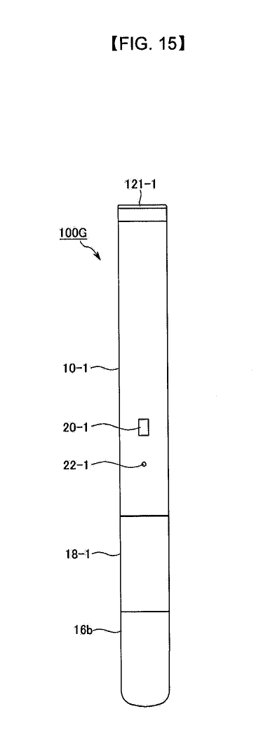

The cap contact portion 16b is shaped to cover the ball contact portion, is connected with the storage portion 182, and massages the surface of the skin. The cap contact portion 16b is separated from the housing 10-1 in FIG. 14, and is connected to the housing 10-1 in FIG. 13 and FIG. 15. In the state of connection of the cap contact portion 16b to the housing 10-1, the surface of the cap contact portion 16b contacts the skin for massage. In this case, the cap contact portion 16b is electrically disconnected from the second contact portion 18-1, and is electrically connected to the ball contact portion 16a.

The second contact portion 18-1 is electrically connected to the battery 12-1, and the second contact portion 18-1 directly contacts the hand of the user when the user holds the iontophoresis device 100G to use it.

At least a part of the second contact portion 18-1 may be made of a transparent material. The storage portion 182-1 may be made of a glass material. A display unit, which displays the left amount of the active material filled in the storage portion 182 may be mounted to a part of the second contact portion 18-1 when the second contact portion 18-1 is made of an opaque material, so that the amount of the active material remaining in the storage portion 182 may be checked.

The storage portion 182 may include an electrically conductive layer that is electrically connected to the ball contact portion 16a between the second contact portion 18-1 and the storage portion 182. The storage portion 182 may be separated from the second contact portion 18-1 and the electrically conductive layer. The second contact portion 18-1 is made of an insulating material.

Hereinafter, an experimental example according to the first exemplary embodiment of the present invention using an iontophoresis device will be described.

### Experimental Example 1

A 5% AA-2G solution that is a vitamin C derivative was coated on liver spots and freckles of skin that was required to be lightened, and the iontophoresis device was used for 10 to 15 minutes every morning and night (i.e., twice a day) to determine improvement of skin troubles including the liver spots and freckles. 25 healthy people between 30 and 59 years of age were tested for 8 weeks. A 6V voltage was used, and the duty cycle was 30%. In addition, a current control button was used so that the skin may not be stimulated. Ultraviolet ray photographs of the freckles were taken to evaluate the following improvement. The iontophoresis device was turned on with respect to a side of each face of one experimental group, and the iontophoresis device was turned off with respect to the other side of each face of a comparison group.

Firstly, testees or families of the testees stated improvement of the freckles after one week of the experiment. In addition, when taking photographs of the skin of the experimental group and the comparison group and comparing them, they showed statistically significant differences in melasma area and severity index (MASI) scores, and therefore the lightening effects were proven (4 weeks: p<0.05, 8 weeks: p<0.005).

Secondly, according to a survey to the testees, as the period of the experiment increased, the number of testees that acknowledged improvement among the experimental group increased.

Thirdly, there were no considerable side effects in the experimental group or the comparison group. However, while the testees of the iontophoresis device felt an itch or a sting, it was gone after the experiment was completed, which has been reported in various papers.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

The iontophoresis device according to the exemplary embodiment of the present invention may concentrate skin care to where liver spots and freckles exist, and may be compact, portable, and convenient.

1. An iontophoresis device for promoting skin to absorb an active material, comprising:
a housing providing an inner space;
a battery installed in the housing for supplying power;
a control circuit for controlling a current output from the battery;
a first contact portion electrically connected with the battery and contacting the skin;
a second contact portion electrically connected with the battery and disposed to the housing; and
an operation portion for turning the power on and off.

2. The iontophoresis device of claim 1, further comprising a storage portion installed to an end of the housing for filling with the active material, wherein the first contact portion is configured to the storage portion, and further comprising a discharging means for discharging the active material filled in the storage portion to the outside thereof.

3. The iontophoresis device of claim 2, wherein the storage portion comprises an outer case, and a storage tank that is made of a transparent material and that is configured inside of the outer case.

4. The iontophoresis device of claim 3, wherein the storage portion comprises an electrically conductive layer that is electrically connected to the first contact portion between the outer case and the storage tank.

5. The iontophoresis device of claim 4, wherein the storage tank can be separated from the outer case and the electrically conductive layer.

6. The iontophoresis device of claim 5, wherein the storage tank is made of a glass material.

7. The iontophoresis device of claim 2, wherein the outer case comprises a display unit, which displays the left amount of the active material in the storage tank.

8. The iontophoresis device of claim 7, wherein the outer case is made of an insulating material.

9. The iontophoresis device of claim 7, wherein the display unit is formed by eliminating a part of the outer case in a slot shape.

10. The iontophoresis device of claim 4, wherein the discharging means is a wick that penetrates the first contact portion, absorbs the active material filled in the storage tank, and releases the active material to the outside thereof.

11. The iontophoresis device of claim 4, wherein the discharging means is an airless pump configured to an opening of the storage tank.

12. The iontophoresis device of claim 2, wherein the iontophoresis device further comprises a cap that is assembled or disassembled with the storage portion for protecting the first contact portion.

13. The iontophoresis device of claim 2, wherein the battery is a coin-type battery, a seat-type battery, or a cylindrical battery.

14. The iontophoresis device of claim 13, wherein an opening/closing portion is formed in the housing for replacing the battery.

15. The iontophoresis device of claim 2, wherein the current control circuit has a function that can control a level of the current flowing from the battery to the skin of a user through the first contact portion.

16. The iontophoresis device of claim 15, wherein the iontophoresis device further comprises an indicating portion that indicates an on/off state of the power and the level of the current.

17. The iontophoresis device of claim 2, wherein the current control circuit has a function such that a pulse-type current can flow to the skin of a user.

18. The iontophoresis device of claim 17, wherein a frequency of the pulse-type current is 1000 to 50,000 Hz.

19. The iontophoresis device of claim 2, wherein the first contact portion is made of stainless steel.

20. The iontophoresis device of claim 19, wherein a coating layer made of titanium nitride (TiN) is formed on a surface of the first contact portion.

21. The iontophoresis device of claim 1, further comprising a storage portion installed to an end of the housing for filling with the active material, and the storage portion comprises an outer case and an electrically conductive layer that is configured inside of the outer case and is electrically connected to the first contact portion, wherein the first contact portion is a ball.

22. The iontophoresis device of claim 1, wherein the storage portion is provided with an opening at an end thereof, and the ball is configured at the opening to spin.

23. The iontophoresis device of claim 1, wherein the active material comprises a material, that has a skin lightening effect.

24. The iontophoresis device of claim 23, wherein the material that has a skin lightening effect is at least one of arbutin, vitamin C (ascorbic acid), MAP (magnesium ascorbyl phosphate), and AA-2G (ascorbyl acid-2 glucoside).

25. The iontophoresis device of claim 1, wherein the active material comprises a material that has a wrinkle care effect.

26. The iontophoresis device of claim 25, wherein the material that has a wrinkle care effect is at least one of vitamin C (ascorbic acid), MAP (magnesium ascorbyl phosphate), AA-2G (ascorbyl acid-2 glucoside), paeoniflorin, kinetin, an auxin, a peptide, and an alpha-hydroxy acid.

27. The iontophoresis device of claim 26, wherein the peptide comprises 10 or fewer amino acids.

28. The iontophoresis device of claim 27, wherein the peptide comprises 2 through 6 amino acids.

29. The iontophoresis device of claim 1, wherein the first contact portion that is formed to be assembled and disassembled is mounted to an end of the housing, and the first contact portion is provided with an internal space for filling with the active material.

30. The iontophoresis device of claim 29, wherein the housing forms a protrusion portion at the end to which the first contact portion connects.

31. The iontophoresis device of claim 30, wherein the protrusion portion is cylindrical in shape.

32. The iontophoresis device of claim 31, wherein a male thread is formed on a circumferential surface of the protrusion portion.

33. The iontophoresis device of claim 32, wherein a female thread corresponding to the male thread of the protrusion portion is formed on an inner surface of the first contact portion, and the first contact portion is connected with the protrusion portion by screw coupling.

34. The iontophoresis device of claim 33, wherein the first contact portion is provided with an opening at an end of the first contact portion, and the first contact portion comprises a ball that is configured in the opening and spins.

35. The iontophoresis device of claim 29, wherein an electrically conductive layer is formed on an inner surface of the first contact portion.

36. The iontophoresis device of claim 29, wherein the first contact portion is made of a conductor.

37. The iontophoresis device of claim 29, wherein the housing has a bar shape.

38. The iontophoresis device of claim 32, wherein the first contact portion comprises:
a main body portion in which a female thread corresponding to the male thread of the protrusion portion is formed on an inner surface thereof, and that is connected with the protrusion portion by screw coupling; and
a head portion that is connected to the main body portion and massages the skin.

39. The iontophoresis device of claim 38, wherein an electrically conductive layer is formed on an inner surface of the main body portion.

40. The iontophoresis device of claim 38, wherein the main body portion is made of a conductor.

41. The iontophoresis device of claim 38, wherein the main body portion and the head portion are integrally formed.

42. The iontophoresis device of claim 32, wherein the first contact portion comprises:
a covering portion identically formed with the male thread of the protrusion portion; and
a body portion in which an upper part connecting with the covering portion is opened, that has an inner space in which a female thread corresponding to the male thread of the covering portion is formed on an inner surface thereof, and that is connected with the covering portion by screw coupling.

43. The iontophoresis device of claim 42, wherein the protrusion portion part is connected to a lower surface of the body portion.

44. The iontophoresis device of claim 42, wherein the protrusion part is cylindrical in shape and has an opening at an end thereof, and the first contact portion comprises a ball that is configured to spin at the opening.

45. The iontophoresis device of claim 44, wherein a male thread is formed on an external circumferential surface of the protrusion part.

46. The iontophoresis device of claim 45, wherein a female thread corresponding to the male thread of the protrusion part is formed, and the iontophoresis device further comprises a protecting cap that is connected with the protrusion part by screw coupling.

47. The iontophoresis device of claim 42, wherein the body portion is made of one of glass, acrylonitrile-butadiene-styrene (ABS), polyethylene (PE), polyethylene terephthalate (PET), and a conductive plastic.

48. The iontophoresis device of claim 42, wherein the body portion is made of glass and an inner side of the body portion is made of a conductive plastic.

49. The iontophoresis device of claim 46, wherein the iontophoresis device further comprises a head portion that is connected to the protecting cap and massages the skin.

50. The iontophoresis device of claim 49, wherein an electrically conductive layer is formed on an inner surface of the protecting cap.

51. The iontophoresis device of claim 49, wherein the protecting cap is made of a conductor.

52. The iontophoresis device of claim 49, wherein the protecting cap and the head portion is integrally formed.

53. The iontophoresis device of claim 1, wherein the iontophoresis device further comprises a storage portion installed in the housing for filling with the active material,
and the first contact portion comprises:
a ball contact portion that is connected with an end of the storage portion and forms a path where the active material filled in the storage portion is released to the outside thereof; and
a cap contact portion that is shaped to cover the ball contact portion, is connected with the storage portion, and a surface thereof massages the skin.

54. The iontophoresis device of claim 53, wherein the ball contact portion comprises:
a connecting portion of which an end thereof is connected with the storage portion;
a ball surface is configured to an opening to spin where the opening is formed at the other end of the connecting portion; and
an elastic portion surface is disposed between an end of the storage portion and the ball and that supports the ball in the connecting portion.

55. The iontophoresis device of claim 54, wherein the storage portion comprises a protrusion portion that is formed in a cylindrical shape by protruding a part connected to the connecting portion, the part is inserted into the connecting portion, and the protrusion portion has an opening to form a path to flow the active material to the outside thereof.

56. The iontophoresis device of claim 55, wherein the iontophoresis device further comprises a guide portion that is safely disposed on the opening of the protrusion portion and supports the elastic portion.

57. The iontophoresis device of claim 56, wherein the guide portion comprises a bottom portion having a groove where the elastic portion is safely disposed, and the bottom portion has a hole for the active material to be released.

58. The iontophoresis device of claim 57, wherein a blocker is formed on a circumference of the guide portion, and the diameter of the blocker is larger than the internal diameter of the opening of the protrusion portion.

59. The iontophoresis device of claim 53, wherein a cap contact portion is made of a conductor.
